(19) United States
(12) Plant Patent Application Publication
(10) Pub. No.: US 2009/0139000 P1
(43) Pub. Date: May 28, 2009

(54) Strawberry plant named 'DrisStrawFive'

(50) ;

(76) Inventors: Tom Rogers, Maidstone (GB); Aloysius G. Claessens, Berkel-Enschot (NL); Thomas M. Sjulin, Aromas, CA (US); Carlos D. Fear, West Malling (GB)

Correspondence Address: JONDLE & ASSOCIATES P.C., 858 HAPPY CANYON ROAD SUITE 230, CASTLE ROCK, CO, 80108, US

(21) Appl. No.: 11/985,923

(22) Filed: Nov. 19, 2007

(51) Int. Cl. A01H 5/00

(52) U.S. Cl. PLT/208

(57) ABSTRACT

This invention relates to a new and distinct cultivar of strawberry plant named ‘DrisStrawFive’. A new cultivar primarily characterized by its small, sweet tasting fruit, moderate resistance to high pH and high soil salt levels, is disclosed.

### GENUS AND SPECIES

Fragaria×ananassa

### VARIETY DENOMINATION

‘DrisStrawFive’

### BACKGROUND OF THE NEW PLANT

The present invention relates to a new and distinct strawberry cultivar designated ‘DrisStrawFive’ and botanically known as Fragaria×ananassa. This new strawberry cultivar was discovered in Kent, England in August, 2002 and originated from a cross between the female parent ‘Driscoll Jubilee’ (U.S. Plant Pat. No. 15,435) and the male parent ‘Driscoll Camarillo’ (U.S. Plant Pat. No. 14,771). The original seedling of the new cultivar was asexually propagated at a nursery in Kent, England. ‘DrisStrawFive’ was subsequently asexually propagated by stolons and underwent further testing at a nursery in Monterey, Calif. for five years. The present invention has been found to retain its distinctive characteristics through successive asexual propagations by stolon.

### DESCRIPTION OF THE PHOTOGRAPHS

The accompanying color photographs show typical specimens of the new cultivar at various stages of development as nearly true as it is possible to make in color reproductions.

FIG. 1 shows overall plant habit including fruit at various stages of development.

FIG. 2 shows leaves of the plant with three leaflets.

FIG. 3 shows both the upperside and underside of several of the flowers.

FIG. 4 shows the whole fruit.

FIG. 5 shows the fruit in longitudinal cross-section.

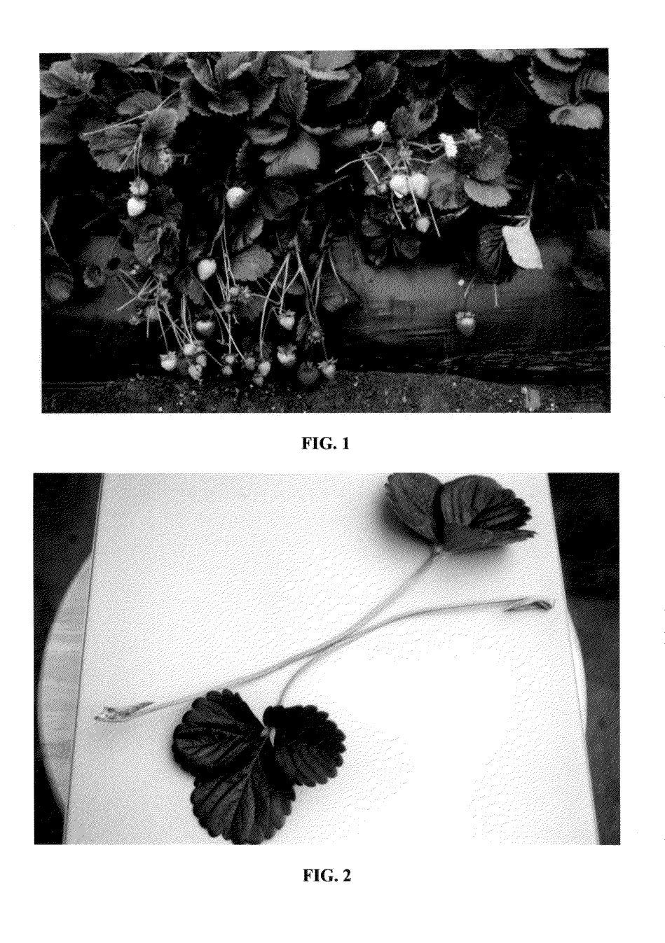

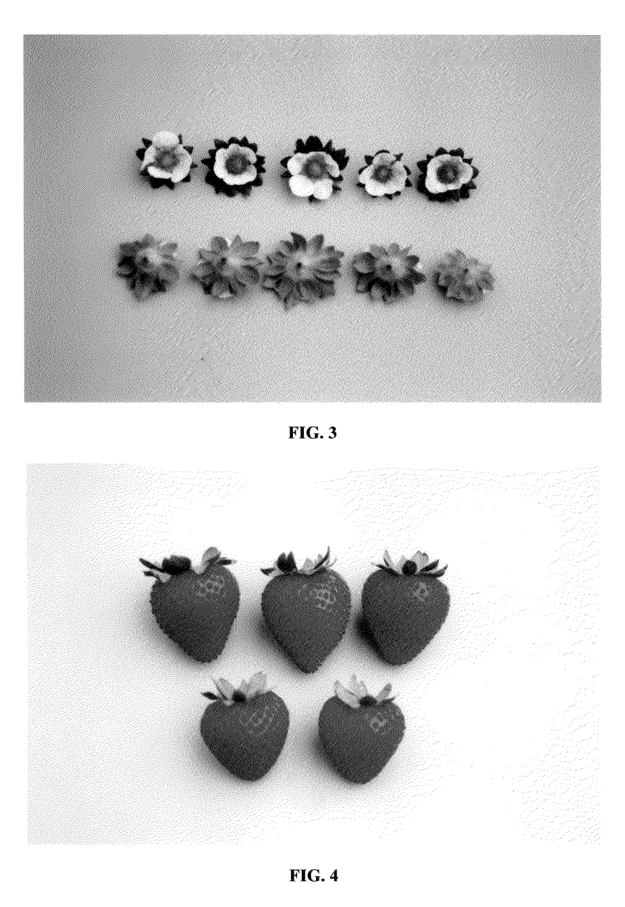

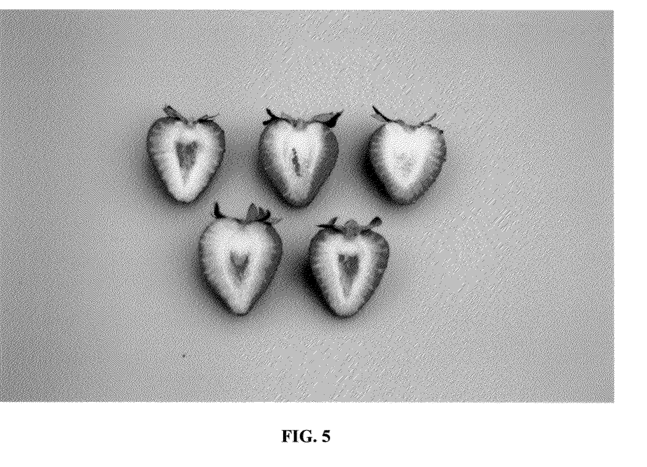

### DESCRIPTION OF THE NEW CULTIVAR

The following description of ‘DrisStrawFive’ is based on observations taken from the 2002 to 2006 growing seasons in Monterey, Calif. This description is in accordance with UPOV terminology. Color designations, color descriptions, and other phenotypical descriptions may deviate from the stated values and descriptions depending upon variation in environmental, seasonal, climatic and cultural conditions. ‘DrisStrawFive’ has not been observed under all possible environmental conditions. Color terminology follows The Royal Horticultural Society Colour Chart, London (R.H.S.) (2001).

### DETAILED BOTANICAL DESCRIPTION

Table 1 shows plant characteristics of the new variety compared with plant characteristics of the female parent ‘Discoll Jubilee’, (U.S. Plant Pat. No. 15,435) and the male parent ‘Driscoll Camarillo’ (U.S. Plant Pat. No. 14,771). Plant characteristics include plant height, petiole length, stipule pubescence, inflorescence position relative to foliage, flower size, diameter of calyx relative to corolla, fruit truss length, and fruit size.

TABLE 1

| | | ‘Driscoll | ‘Driscoll |
|---|---|---|---|
| Characteristic | ‘DrisStrawFive’ | Jubilee’ | Camarillo’ |
| Plant height (cm) | 31.2 | 25.7 | 27.2 |
| Petiole length | 22.2 | 16.3 | 16.8 |
| (cm) | | | |
| Stipule | Dense | Between sparse | Sparse |
| pubescence | | and medium | |
| Inflorescence | Between level | Beneath | Above |
| position relative | with | | |
| to foliage | and above | | |
| Flower size | Small | Between small | Medium |
| | | and medium | |
| Diameter of calyx | Between | Between | Between |
| relative to corolla | same size | smaller and | smaller and |
| | and larger | same size | same size |
| Fruit truss length | 31.7 | 19.6 | 27.6 |
| (cm) | | | |
| Fruit size | Small | Between small | Between medium |
| | | and medium | and large |

Table 2 shows plant characteristics of the new variety compared with plant characteristics of the commercial varieties ‘Driscoll Jubilee’ (U.S. Plant Pat. No. 15,435) and ‘Driscoll Camarillo’ (U.S. Plant Pat. No.14,771). Plant characteristics include plant height, diameter, number of crowns per plant, habit, density of individual plant and vigor.

TABLE 2

| | | | ‘Driscoll |
|---|---|---|---|
| Characteristic | ‘DrisStrawFive’ | ‘Driscoll Jubilee’ | Camarillo’ |
| Plant height (cm) | 31.2 | 25.7 | 27.2 |
| Plant diameter | 50.0 | 44.5 | 49.7 |
| (cm) | | | |
| Number of | 4 | 4 | 5 |
| crowns/plant | | | |
| Habit | Globose | Flat globose | Globose |
| Density of | Medium | Between open | Open |
| individual plant | | and medium | |
| Vigor | Strong | Between weak | Medium |
| | | and medium | |

Table 3 shows leaf characteristics of the new cultivar compared with leaf characteristics of ‘Driscoll Jubilee’ and ‘Driscoll Camarillo’. Leaf characteristics include terminal leaflet length and width in centimeters, length to width ratio, number of teeth per terminal leaflet, shape of teeth, color of upperside and underside of leaf, leaf shape in cross section, leaf blistering, leaf glossiness, number of leaflets, terminal leaflet margin, terminal leaflet length to width ratio and shape of leaf base.

TABLE 3

| Leaf | | ‘Driscoll | ‘Driscoll |
|---|---|---|---|
| Characteristic | ‘DrisStrawFive’ | Jubilee’ | Camarillo’ |
| Terminal | 0.72 | 0.75 | 0.71 |
| leaflet | | | |
| length (cm) | | | |
| Terminal | 0.66 | 0.68 | 0.75 |
| leaflet | | | |
| width (cm) | | | |
| Terminal | 1.09 | 1.10 | 0.95 |
| leaflet | | | |
| length/width | | | |
| ratio | | | |
| No. teeth/ | 17 | 18 | 23 |
| terminal | | | |
| leaflet | | | |
| Shape of teeth | Rounded | Rounded | Rounded |
| Color of | RHS 139A | RHS 147A | RHS 147A |
| upperside | Dark green | Dark yellow- | Dark yellow- |
| of leaf | | green | green |
| Color of | RHS N138C | RHS 138B | RHS 138B |
| underside | Medium greyed- | Medium green | Medium |
| of leaf | green | | green |
| Leaf shape in | Flat | Between slightly | Concave |
| cross section | | concave and flat | |
| Leaf blistering | Strong | Strong | Very strong |
| Leaf glossiness | Weak | Medium | Between |
| | | | medium |
| | | | and strong |
| No. leaflets | Three only | Three only | Three only |
| Terminal leaflet | Revolute | Revolute | Between |
| margin | | | revolute |
| | | | and flat |
| Terminal leaflet: | Between as long as | Between as long | Between |
| length/width ratio | broad and longer | as broad and | broader than |
| | than broad | longer than broad | long and |
| | | | as long as |
| | | | broad |
| Terminal leaflet | Rounded | Acute | Rounded |
| base shape | | | |

Table 4 shows information about the petiole, the petiolule, the bract, and the stipule of the new cultivar compared to ‘Driscoll Jubilee’ and ‘Driscoll Camarillo’. This includes petiole length in centimeters, petiole diameter in centimeters, petiole pubescence, pose of hairs on the petiole, color of the petiol, color of the petiolule, petiolule length in centimeters, petiolule diameter in centimeters, bract frequency per petiole, stipule length in centimeters, stipule width in centimeters, and stipule pubescence.

TABLE 4

| | | ‘Driscoll | ‘Driscoll |
|---|---|---|---|
| Characteristic | ‘DrisStrawFive’ | Jubilee‘ | Camarillo’ |
| Petiole | 22.2 | 16.3 | 16.8 |
| length (cm) | | | |
| Petiole | 0.256 | 0.354 | 0.331 |
| diameter (cm) | | | |
| Petiole | Dense | Absent or | Sparse |
| pubescence | | very sparse | |
| Petiole pose | Outward | Between outward | Outward |
| of hairs | | and downward | |
| Petiole color | RHS 145A | RHS 145A | RHS 149A |
| | Light yellow- | Light yellow- | Bright yellow- |
| | green | green | green |
| Petiolule | RHS 144A | RHS 146C | RHS 144B |
| color | Medium yellow- | Dark yellow- | Medium yellow- |
| | green | green | green |
| Petiolule | 1.004 | 1.172 | 1.373 |
| length (cm) | | | |
| Petiolule | 0.156 | 0.181 | 0.194 |
| diameter (cm) | | | |
| Bract | 0 | 0 | 0 |
| frequency | | | |
| Stipule | 3.5 | 3.4 | 4.0 |
| length (cm) | | | |
| Stipule | 0.765 | 0.978 | 0.866 |
| width (cm) | | | |
| Stipule | Dense | Between sparse | Sparse |
| pubescence | | and medium | |

Table 5 shows stolon characteristics of the new cultivar compared to ‘Driscoll Jubilee’ and ‘Driscoll Camarillo’. These characteristics include the number of stolons, average number of daughter plants, the anthocyanin coloration of the stolons, the thickness of the stolons, and the pubescence of the stolons.

TABLE 5

| | | ‘Driscoll | ‘Driscoll |
|---|---|---|---|
| Characteristic | ‘DrisStrawFive’ | Jubilee‘ | Camarillo’ |
| Stolon Number | Medium | Medium | Medium |
| Average number | 13 | 45 | 40 |
| of daughter | | | |
| plants | | | |
| Stolon | Strong | Medium | Medium |
| Anthocyanin | RHS 59D Medium | | |
| | Red-purple | | |
| Stolon | Thin | Between thin | Between |
| Thickness | | and medium | medium |
| | | | and thick |
| Stolon | Sparse | Between sparse | Medium |
| Pubescence | | and medium | |

Table 6 shows inflorescence characteristics of the new cultivar compared to ‘Driscoll Jubilee’ and ‘Driscoll Camarillo’. These characteristics include inflorescence position relative to foliage, relative flower size, flower diameter in centimeters (measured from petal tip to petal tip), relative spacing of petals, petal length in centimeters, petal width in centimeters, petal length to width ratio, petal color, calyx diameter in centimeters (measured on back of flower from sepal tip to sepal tip), diameter of calyx relative to corolla, diameter of inner calyx relative to outer, sepal length in centimeters (measured from sepal tip to point of attachment to receptacle), sepal width in centimeters, receptacle color and anther color.

TABLE 6

| | | ‘Driscoll | ‘Driscoll |
|---|---|---|---|
| Characteristic | ‘DrisStrawFive’ | Jubilee’ | Camarillo’ |
| Inflorescence | Between level | Beneath | Above |
| position relative | with and | | |
| to foliage | above | | |
| Flower size | Small | Between small | Medium |
| | | and medium | |
| Flower | 2.174 | 2.299 | 2.698 |
| diameter (cm) | | | |
| Petal spacing | Overlapping | Between | Overlapping |
| | | touching and | |
| | | overlapping | |
| Petal | 1.054 | 1.192 | 1.275 |
| length (cm) | | | |
| Petal | 1.035 | 1.142 | 1.367 |
| width (cm) | | | |
| Petal length/ | As long | As long | Between broader |
| width ratio | as broad | as broad | than long |
| | | | and as long |
| | | | as broad |
| Petal length/ | 1.02 | 1.04 | 0.93 |
| width ratio | | | |
| Petal color | RHS 155C | RHS 155C | RHS 155B |
| | White | White | White |
| Calyx | 2.726 | 2.910 | 3.233 |
| diameter (cm) | | | |
| Calyx diameter | Between | Between | Between |
| relative | same size | smaller and | smaller and |
| to corolla | and larger | same size | same size |
| Inner calyx | Larger | Smaller | Same size |
| diameter | | | |
| relative to | | | |
| outer | | | |
| Sepal | 0.957 | 1.098 | 1.118 |
| length (cm) | | | |
| Sepal | 0.626 | 0.540 | 0.709 |
| width (cm) | | | |
| Receptacle | RHS 1B | RHS 1B | RHS 1A |
| color | Bright green- | Bright green- | Bright green- |
| | yellow | yellow | yellow |
| Anther | RHS 23A | RHS 23A | RHS 17A |
| color | Bright yellow- | Bright yellow- | Dull yellow- |
| | orange | orange | orange |

Table 7 shows fruit characteristics of the new cultivar compared to ‘Driscoll Jubilee’ and ‘Driscoll Camarillo’. These characteristics include fruiting truss length in centimeters, fruiting truss attitude, fruiting truss length, fruit length in centimeters, fruit truss width in centimeters, fruit length to width ratio, fruit weight in grams, relative fruit size, predominant fruit shape, difference in shape between primary and secondary fruits, band without achenes, unevenness of fruit surface, fruit skin color, evenness of fruit color, fruit glossiness, insertion of achenes, achene coloration (sunward and shaded sides of berry), achenes per berry, achene weight in grams, insertion of calyx, pose of calyx segments, size of calyx in relation to fruit, adherence of calyx, firmness of flesh, color of the fruit flesh, evenness of the flesh color, distribution of flesh color, hollow center, sweetness of fruit, acidity of fruit, texture of fruit when tasted, time of flowering, harvest maturity, type of bearing, grams of fruit per plant

TABLE 7

| | | ′Driscoll | ′Driscoll |
|---|---|---|---|
| Characteristic | ′DrisStrawFive′ | Jubilee′ | Camarillo′ |
| Fruiting truss | 31.7 | 19.6 | 27.6 |
| length (cm) | | | |
| Fruiting truss | Semi- | Semi- | Prostrate |
| attitude | erect | erect | |
| Fruiting truss | Long | Medium | Long |
| length | | | |
| Fruit | 3.52 | 3.59 | 3.59 |
| length (cm) | | | |
| Fruit | 3.28 | 3.07 | 3.29 |
| width (cm) | | | |
| Fruit length/ | 1.07 | 1.17 | 1.09 |
| width ratio | | | |
| Fruit length/ | Between as long | Longer than | Between as |
| width ratio | broad and longer | broad | long as broad |
| | than broad | | and longer than |
| | | | broad |
| Fruit weight (g) | 19.4 | 17.3 | 20.6 |
| Relative | Small | Between small | Between |
| fruit size | | and medium | medium and |
| | | | large |
| Predominant fruit | Conical | Conical | Cordate |
| shape | | | |
| Difference in | None or very | Between none | Slight |
| shape between | slight | or very slight | |
| primary & | | and slight | |
| secondary fruits | | | |
| Band without | Absent or | Absent or | Absent or |
| achenes | very narrow | very narrow | very narrow |
| Unevenness of | Absent or | Absent or | Weak |
| fruit surface | very weak | very weak | |
| Fruit skin | RHS 45A | RHS 45A | RHS 46A |
| color | Bright red | Bright red | Dark red |
| Evenness of | Even | Slightly | Even |
| fruit color | | uneven | |
| Fruit | Strong | Strong | Strong |
| glossiness | | | |
| Insertion of | Level with | Between level | Below surface |
| achenes | surface | with surface | |
| | | and above | |
| | | surface | |
| Achene | RHS 185A | RHS 184A | RHS 184A |
| coloration- | Medium greyed- | Medium | Medium |
| sunward | purple | greyed- | greyed- |
| side of berry | | purple | purple |
| Achene | RHS 7D | RHS 4A | RHS 1 3B |
| coloration- | Medium | Bright | Medium |
| shaded side | yellow | medium | orange- |
| of berry | | yellow | yellow |
| Achenes per | 164.6 | 131.3 | 224.0 |
| berry | | | |
| Achene | 0.000499453 | 0.000616527 | 0.000626027 |
| weight (grams) | | | |
| Insertion of | Level | Between | In a basin |
| calyx | | in a basin | |
| | | and level | |
| Pose of | Between | Spreading | Reflexed |
| calyx | spreading | | |
| segments | and | | |
| | reflexed | | |
| Size of calyx | Between | Between | Smaller |
| in relation | same size | smaller and | |
| to fruit | and larger | same size | |
| Adherence | Strong | Strong | Strong |
| of calyx | | | |
| Firmness | Firm | Between | Firm |
| of flesh | | medium and | |
| | | firm | |
| Color of | RHS 155D | RHS 43A | RHS 34B |
| the flesh | White and | Bright red | Medium orange |
| | RHS 41C | and RHS 155B | red and RHS |
| | Very light | White | 155A White |
| | red | | |
| Evenness of | Uneven | Slightly | Slightly |
| flesh color | | uneven | uneven |
| Distribution | Only | Marginal | Marginal |
| of flesh | marginal | and | and |
| color | | central | central |
| Hollow | Between | Between | Between |
| center | absent and | absent and | absent and |
| | small | small | small |
| Sweetness | Between | Between | Medium |
| | medium | medium and | |
| | and strong | strong | |
| Acidity | Medium | Medium | Medium |
| Texture | Medium | Medium | Medium |
| when tasted | | | |
| Time of | Medium | Medium | Medium |
| flowering | | | |
| Harvest | Late April | Early May | Late |
| maturity (50% | to early | to late | September |
| of plants with | November | October | to late |
| ripe fruit) | | | December |
| Type of | Fully | Fully | Fully |
| bearing | everbearing | everbearing | everbearing |
| Grams of | 518 | 979 | 1,485 |
| fruit/plant | | | |

Table 8 shows the resistance to different forms of stress of the new cultivar compared to ‘Driscoll Jubilee’ and ‘Driscoll Camarillo’. These forms of stress include high pH and high soil salt levels.

TABLE 8

| Reaction to | | ′Driscoll | ′Driscoll |
|---|---|---|---|
| Stress | ′DrisStrawFive′ | Jubilee′ | Camarillo′ |
| High pH | Moderately | Moderately | Moderately |
| | resistant | resistant | resistant |
| High soil | Moderately | Moderately | Moderately |
| salt levels | resistant | susceptible | resistant |

Table 9 shows pest and disease characteristics of the new cultivar compared to ‘Driscoll Jubilee’ and ‘Driscoll Camarillo’.

TABLE 9

| | | ′Driscoll | ′Driscoll |
|---|---|---|---|
| Pest or Disease | ′DrisStrawFive′ | Jubilee′ | Camarillo′ |
| Tetranychus urticae | Moderately | Moderately | Moderately |
| (2-spotted spider | susceptible | susceptible | susceptible |
| mite) | | | |
| Tarsonemus pallidus | Susceptible | Susceptible | Susceptible |
| Botrytis fruit rot | Moderately resistant | Moderately | Moderately |
| | to moderately | resistant to | resistant |
| | susceptible | moderately | to moderately |
| | | susceptible | susceptible |
| Powdery mildew | Susceptible | Susceptible | Between |
| | | | susceptible |
| | | | and highly |
| | | | susceptible |
| Verticillium wilt | Moderately resistant | Moderately | Moderately |
| | to moderately | resistant | susceptible |
| | susceptible | | |

Table 10 shows overall characteristics of the new cultivar compared to ‘DrisStrawOne’ (U.S. Plant Pat. No. 18,458). These characteristics include the plant height and diameter in centimeters, petiole length and diameter in centimeters, fruit length and width in centimeters; fruit sweetness and fruit texture when tasted.

TABLE 10

| Characteristic | ′DrisStrawFive′ | ′DrisStrawOne′ |
|---|---|---|
| Height (cm) | 31.2 | 9.5 |
| Diameter (cm) | 50.0 | 15.9 |
| Petiole length (cm) | 22.2 | 12.5 |
| Petiole diameter (cm) | 0.256 | 0.385 |
| Fruit length (cm) | 3.52 | 0.538 |
| Fruit width (cm) | 3.28 | 0.452 |
| Sweetness | Between medium | Medium |
| | and strong | |
| Texture when tasted | Medium | Fine |

1. A new and distinct cultivar of strawberry plant as described and shown herein.
